(19) United States
(12) Patent Application Publication
(10) Pub. No.: US 2020/0263138 A1
(43) Pub. Date: Aug. 20, 2020

(54) METHOD FOR PRODUCING A THREE-DIMENSIONAL BIOLOGICAL STRUCTURE AND SAID STRUCTURE THUS OBTAINED

(71) Applicant: RHEINISCH-WESTFALISCHE TECHNISCHE HOCHSCHULE AACHEN (RWTH), Aachen (DE)

(72) Inventor: Andreas BLAESER, Troisdorf (DE)

(21) Appl. No.: 16/642,657

(22) PCT Filed: Sep. 6, 2018
(86) PCT No.: PCT/EP2018/073960

§ 371 Date: Feb. 27, 2020

(30) Foreign Application Priority Data
Sep. 6, 2017 (DE) 10 2017 120 470.9

(51) Int. Cl. C12N 5/071

(52) U.S. Cl. CPC C12N 5/0691; C12N 5/0671; C12N 5/0686; C12N 2533/76; C12N 2533/54; C12N 2533/56; C12N 2513/00

(57) ABSTRACT

The invention relates to a mctliod for bio-printing a thrcc-dimensional biological structure containing liv ing cells having at least two different materials for the bio-printing. Said method is distinguished by the fact that, in at least one step, one of the materials for printing is applied or introduced by printing droplets (drop-on-dentand) printing. In particular, this method is suitable for printing tissue structures. including those which have supply structures. Such structures are in particular cardiac structures, liver structures, kidney structures, alveolar structures, skin struchircs or neural structuies. The invention further relates to a biological three-dimensional structure thus obtainable. Finally, the invention relates to the use of a three-dimensional structure according to the invention as a tissue model, in particular as a model for tissue genesis, for example suitable for testing therapy forms or for the stratification of a therapy or for testing or identifying active substance candidates.

The present application relates to a process for bioprinting a three-dimensional biological structure containing live cells using at least two different materials for bioprinting. Said process is notable in that, in at least one step, one of the materials for printing is applied or introduced by printing of droplets (drop-on-demand). Said process is especially suitable for printing tissue structures, including those which have supply structures. Such structures are, in particular, cardiac structures, liver structures, kidney structures, alveolar structures, skin structure or neural structures. In a further aspect, a thus obtainable biological three-dimensional structure is described. Lastly, the present application relates to the use of a three-dimensional structure according to the invention as a tissue model, especially as a model for tissue genesis, for example suitable for testing forms of therapy or for stratifying a therapy or for testing or identifying active-ingredient candidates.

### PRIOR ART

Many tissues, which are present in the human body for example, comprise a multiplicity of cell types which are surrounded by matrices of differing physical and chemical composition. Examples that may be mentioned here are the liver, the kidney or the myocardium. In these tissues, matrices and cell types cannot be found in a uniform distribution; instead, they have a high degree of microstructural organization, i.e., a highly organized microstructure. Important to this is, inter alia, the presence of a capillary network; this supplies the relevant nutrients and the respiratory gases and removes relevant metabolic degradation products. The technology of bioprinting, which has been the subject of research for some years, has the potential to copy the complex structure of the stated tissues up to a certain degree, with the goal of using said tissues as implant or as in vitro screening platform for active pharmacological ingredients and for toxicity tests (Murphy S. V., Atala A. Nat. Biotechnol, 2014, 32(8), 773-785, Blaeser A., et al., Curr. Opin. Biomed. Eng. 2017, doi:10.1016/j.cobme.2017.04.003).

Bioprinting is a specific tissue-engineering process which makes use of the principle of additive manufacturing processes—also known as rapid prototyping—in order to generate three-dimensional live tissues. In contrast to additive manufacturing processes used in industry, such as, for example, stereolithography, selective laser melting or fused deposition modeling, what are typically used as construction material in the case of bioprinting are printable materials loaded with cells, for example hydrogels. Here, this material is usually applied layer by layer according to a 3D model, with the hydrogels imitating the extracellular matrix of the natural tissue and, in doing so, forming an appropriate cell-friendly environment for 3D tissue engineering.

Current approaches for bioprinting pursue the goal of reconstructing the anatomy and shape of a desired tissue type as realistically as possible. The consequential complexity requires that the particular 3D model, the software used, the hardware used, the biomaterial and the printing strategy need to be specifically tailored to the target tissue. This gives rise to a multiplicity of highly specific bioprinting approaches which allow the printing of only one specific tissue type. Different strategies and modalities are required for the appropriate formation of various tissue types. For example, it is described in the literature that photopatterning can be used to generate liver models and that microextrusion processes are used to form kidney models, skin models or cartilage and bone models. Coaxial microextrusion has been used in conjunction with crosslinking by UV light irradiation in order to form bioprinted thrombosis models or a vascularized myocardium. There is a large multitude of possible production processes, production strategies, biomaterials and printing software, which are not only time-consuming, but also inefficient and inflexible for possible standard use and for attainment of a certification of said production processes, which is required in the area of implants. Accordingly, commercial realization is difficult.

The publication by Stratesteffen H., et al., Biofabrication 9 (2017) 045002, describes the printability by drop-on-demand 3D printing for GelMA-collagen blends and their promotion of angiogenesis. In Scientific Reports, 6:39140/D01:10.1038/SREP39140, Tan, Y. J. et al. describe hybrid microscaffold-based 3D bioprinting of multicellular constructs with high compressive strength. Similar to in Stratesteffen, what are described therein are novel composite materials for the purpose of 3D printing.

Marchioli, G. et al., DOI:10.1002/ADHM.201600058, disclose a process which describes a hybrid PCL scaffold filled with cell-loaded hydrogel. Here, the PCL is extruded with extrusion at 100° C.

An overview of 3D bioprinting processes, especially those for printing hydrogels comprising live cells for tissue generation, is discussed in the article by Blaeser et al., 2017; see above. The different ways of 3D bioprinting are described therein. A distinction is made between processes which allow a layer-by-layer construction, a line-by-line construction and a droplet-by-droplet construction. By applying various techniques, it is possible to construct a very wide variety of different structures which, as already explained above, are usually tissue-specific.

Droplet-based bioprinting can be carried out with different techniques; possibilities here include the laser-based process, inkjet printing and the microvalve process. The processes differ in resolution or droplet size, in the achievable cell viability, and in suitability for printing planar microstructures and freestanding 3D structures.

As explained, various techniques can be used in bioprinting; one of these printing processes is that by means of droplets (droplet printing) using the three techniques that are mentioned above in principle, the techniques of laser-based printing, inkjet printing or printing by means of microvalves.

The advantages of these printing techniques are the formation of individual droplets which can comprise live cells if necessary, with the possible size thereof being up to 0.01 nl in volume; the current resolution is up to 45 μm.

The individual droplets can be applied to predetermined positions in a simple and highly accurate manner. As a result, it is possible to apply highly precise structures. This is especially the case when using multiple print heads, these making it possible to use different printing materials.

There are thus many processes for bioprinting and for producing tissue structures, though they have different disadvantages, in particular the hitherto unfeasible general formation of various types of tissue structure.

Accordingly, there is a need to provide improved processes which can provide three-dimensional biological structures containing live cells by means of bioprinting on the basis of a general principle, with these various tissue structures being achievable in a simple and cost-effective manner. By means of one simple method, structural hierarchies of multiple tissue types are generated using a novel bioprinting approach.

### BRIEF DESCRIPTION OF THE INVENTION

According to the invention, it is possible to use a simple technique and a simple process to generate three-dimensional biological structures which reflect diverse tissue types when generated in vitro.

The most important difference in relation to previously mentioned bioprinting strategies is that priority is given not to the shape and anatomy of the target tissues, but to the biological function thereof. What is attempted is not to copy the anatomy of an individual tissue in a one-to-one manner, but to identify the anatomically lowest common denominator of a largest possible number of different tissues and to reproduce it by means of bioprinting.

In a first aspect, the present application provides a process for bioprinting a three-dimensional biological structure containing live cells using at least two different materials for bioprinting that form, in line with their material properties, at least two different subregions, wherein at least one subregion of said three-dimensional structure contains live cells and wherein at least one of the materials for bioprinting contains live cells, wherein one of said materials is applied or introduced to a substrate in a first step, said material is optionally subjected to a first treatment, especially a wash, and a material other than the one used in the first step is applied or introduced in a subsequent step, characterized in that at least one of the materials is applied or introduced by droplet printing.

This process according to the invention can, for example, be carried out as an infiltration method or as a penetration method.

Furthermore, the present invention provides biological three-dimensional structures obtainable using said method, these being especially tissue structures and tissue models.

Said tissue models are suitable for use, for example, of tissue genesis or as an in vitro model for testing forms of therapy or for stratifying the therapy or for testing or identifying active-ingredient candidates.

### DESCRIPTION OF THE FIGURES

FIG. 1 depicts schematically the infiltration and penetration methods that are described herein.

FIG. 1a shows the infiltration method. Droplets of a first printing material (bioink A) are printed onto a substrate. This is followed by a washing/rinsing, for example with PBS, of the material applied as droplets so that the second printing material (bioink B) can then be applied in order to thus fill up the spaces between the bioink A.

The steps of applying droplets of the bioink A, washing and applying the bioink B can be repeated multiple times if necessary in order to obtain a multicellular tissue model.

FIG. 1b depicts the penetration method. To this end, the printing material bioink B is applied, for example by spraying, dripping, etc., to a substrate which has been washed with PBS. Thereafter, the printing material bioink A is printed on as droplets, with said droplets penetrating into the film formed by the bioink B. These steps can be repeated multiple times if necessary in order to obtain multicellular tissue models.

FIG. 1c depicts the various multicellular tissue models. The left-hand side depicts a composite structure with bioink A and surrounding bioink B. The center depicts a porous mesh composed of the bioink B. The bioink A which was formerly present has been leached, for example by dissolving of the corresponding printing material by means of, for example, water, heating, etc., and thus liquefaction.

The right-hand side depicts a porous composite structure. In this case, the bioink A has been taken out in the central region in order to obtain a cavity. In said cavity, the remaining cells can accordingly be present on the wall. Vascular structures can be created as a result. The bioink A is present in the remaining regions with encapsulated cells. Alternatively, it is also possible to introduce multicellular spheroids (MCS) into the cavities.

FIG. 2a shows the results of the infiltration technique (left) and penetration technique (right). FIG. 2b depicts a capillary-type network formed as per FIG. 2a by infiltration. The left-hand part of FIG. 2b depicts transmitted-light images (“BF”) of microscopic scans of the control (cells in unstructured fibrin) and a printed sample. A vital-fluorescence twin staining shows the proportion of live (“Live”, originally green) and dead cells (“Dead”, originally red). The right-hand part depicts subregions of the prior images at stronger magnification. Moreover, a negative control is depicted, in which a control sample was treated with ethanol, the result being that the cells were killed and it is possible to show that the measurement method also captures dead cells. FIG. 2c depicts details of the printed structure. What can be seen are the three-dimensional structures which are accordingly formed. After two weeks of culture, capillary-type networks have formed owing to the endothelial cells. Supply structure-type regions with corresponding cavities can be clearly seen, as can other regions with cell structures. To depict the vascular network, an immunofluorescence staining with DAPI (nucleus, originally blue) and CD31 (endothelial cell-specific marker, originally red) was carried out and recorded by means of two-photon microscopy (bottom, right) and fluorescence microscopy (bottom, left).

FIG. 3 depicts an application according to the invention for generating three different tissue structures. FIG. 3a shows, by way of example, the generation of a cardiac muscle precursor structure. Here, cardiomyocytes were embedded in fibrin. Just after a few days in culture, the unprinted control exhibited considerable deformations, caused by contraction of the cardiac muscle cells. In the case of the printed structure, agarose droplets were printed by means of the penetration method into the fibrin layer, which mechanically stabilizes the structure during cell growth. It can be easily seen that the structures were not deformed or were only negligibly deformed after 5 and even 7 days after production and that the cells aligned themselves especially along the printed structures. FIG. 3b depicts the application of the described technique for generating a kidney tissue, specifically the renal tubulointerstitium. Here, tubuloepithelial cells were embedded in droplets of a leachable porogen (agarose-gelatin mixture). The spaces between the printed droplets were infiltrated with fibrin which contained human mesenchymal stem cells (hMSCs) and human umbilical vein endothelial cells (HUVECs). After the leaching of the porogen, epithelial cells adhered on the edge of the resultant pores, where they formed a homogeneous cellular layer. After seven days of culturing, the hMSCs and HUVECs present in the surrounding fibrin formed first capillary-like structures. The printed structures were stained and were examined by means of fluorescence microscopy and two-photon microscopy. To this end, all cells were labeled with DAPI, the endothelial cells additionally with CD31, the epithelial cells with LTL antibody stains. The mesenchymal stem cells are visible by autofluorescence in the two-photon microscopy. FIG. 3c depicts the application of the technique according to the invention for generating a liver tissue, specifically a liver lobe tissue. For this purpose, hepatocytes were embedded in agarose droplets, which were printed by means of the penetration method into a fibrin layer loaded with hMSCs and HUVECs, which construct a capillary-like network during their culturing. By repeating multiple times, a structure comprising six layers was produced. In the micrograph, the hepatocytes can be distinguished from the remaining cells on the basis of their morphology. For better depiction, they were virtually colored with the aid of an algorithm. The amount of synthesized urea was measured over 12 days on the thus produced liver lobe tissues (HEP+) and compared with a further sample in which the hepatocytes were printed without a supporting capillary network (HEP). Hepatocytes having a surrounding capillary network exhibited a significantly higher level of urea synthesis than the comparison group and the control (only cell culture medium).

FIG. 4: FIG. 4 shows that the structures obtained from the printing technique can also be combined with multicellular spheroids (MCS), depicted in FIG. 4a. Said MCS can either be directly coprinted in the droplets, as depicted in FIG. 4b, or they can be embedded in the resultant pores after the leaching of the droplets; see FIG. 4c.

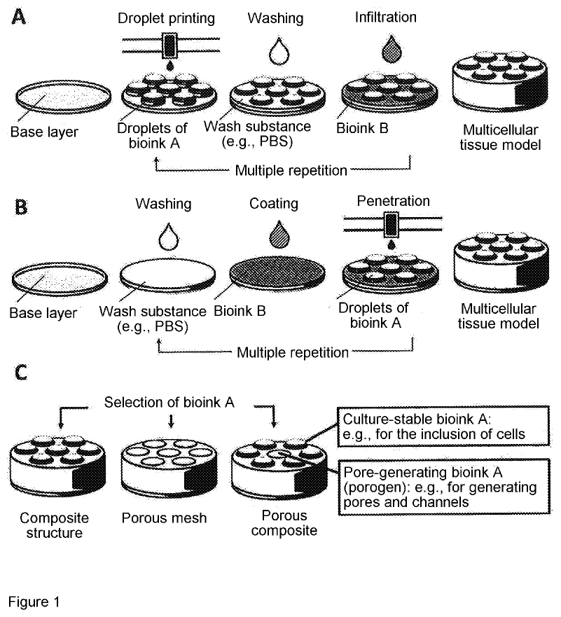

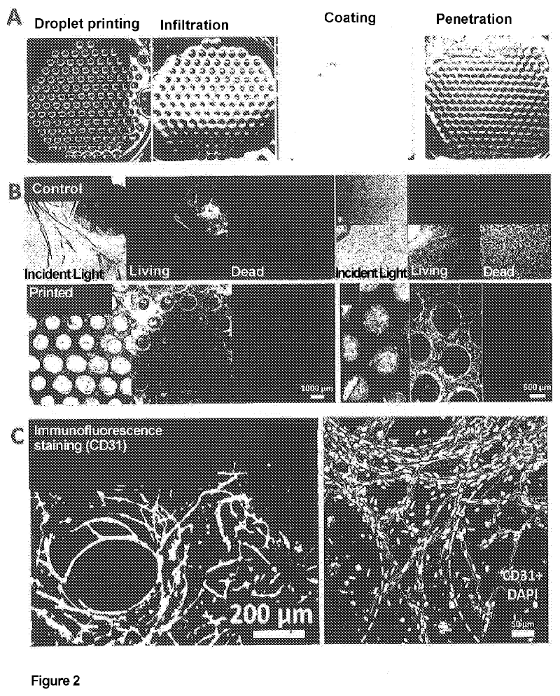

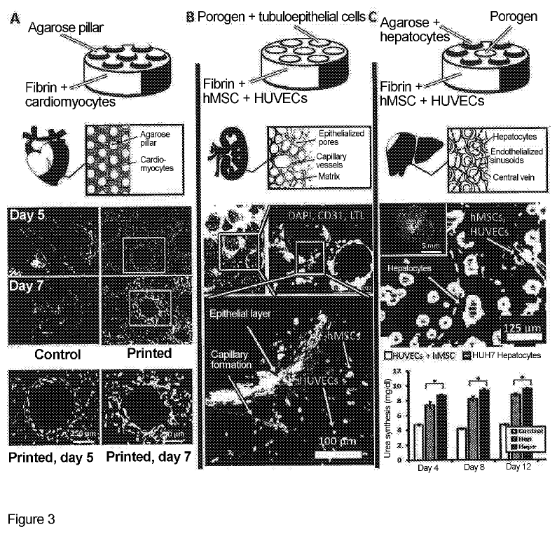

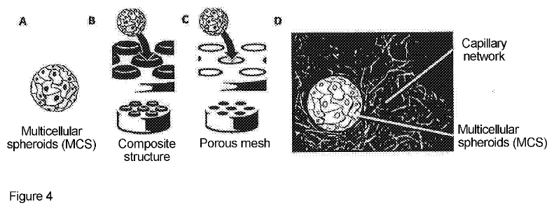

Accordingly, complex structures can be produced, such as, for example, those comprising MCS connected via a capillary network; by way of example, see FIG. 4d. Here, suitable spheroids can consist of stem cells, adult cells, primary cells, knockout cells, wild-type cells, degenerate cells or tumor cells or combinations of such cells.

### DETAILED DESCRIPTION OF THE INVENTION

The inventors have succeeded in providing a process which, on the basis of general process steps, allows the generation of different three-dimensional biological structures, especially tissue structures, which have an irregular construction, in particular do not have regularly distributed structures, but have instead regions with supply structures and other specific regions.

This means that the process according to the invention is a platform process which can generate a multiplicity of tissue analogs by printing corresponding irregular structures in their preform. It is possible by means of this drop-on-demand (DOD) based bioprinting process to construct the structural hierarchy of multiple tissue types. The process according to the invention differs from the prior art in that, firstly, the described process has not been previously described for tissue model formation and, secondly, the process according to the invention utilizes the basic principles of structural similarities, which are generally revealed in different tissue structures.

In contrast to the prior-art processes, which specifically construct one tissue structure, the process according to the invention makes it possible to depict tissue anatomy in relatively abstract form and to generate it by culturing. Thus, under appropriate conditions, it is possible on the basis of these general structures with use of the tissue structure-specific cells and possibly culturing conditions to copy the individual tissue structure.

Through this general basic principle of the process according to the invention, it is possible to provide completely different tissue structures as analogs with high biofunctional similarity to their natural counterparts.

This means that the printing process is essentially identical for a very wide variety of different tissue structures and that only the relevant printing materials are different, i.e., they comprise different cell types and/or MCS and possibly comprise different matrix materials.

Using the process according to the invention, it is thus possible to produce different tissue models in a simple and reproducible manner. The process has a high degree of flexibility and is a process which is industrially implementable and which can meet the authorization requirements for the medical sector.

The process according to the invention, wherein at least one of the materials is applied using a droplet-based printing process, especially drop-on-demand (DOD) bioprinting, is one in which separate droplets containing matrix materials which comprise or do not comprise cells, also referred to here as bioink, is printed in order to provide a three-dimensional biological structure, especially a tissue-structure analog.

In the most general approach, at least one first material, also referred to as “one material”, and at least one second material, also referred to as “other material”, are used in order to form, respectively, a first subregion and a second subregion of this three-dimensional structure. One of said materials comprises live cells, meaning that live cells are present in at least one of said subregions.

Unless otherwise stated, the term “cell” or “cells” also encompasses multicellular spheroids (MCS). MCS can, for example, consist of or comprise: stem cells, adult cells, primary cells, knockout cells, wild-type cells, degenerate cells or tumor cells or combinations thereof.

According to the general process, one material is applied to a substrate in a first step. This is followed by the application of a further or other material. Said application can be done in various different ways. Preferred embodiments are depicted in the figures.

In one embodiment, the one material as first material is printed onto a substrate as droplets in the first step, wherein said droplets have predetermined positions in relation to one another, and then, optionally after a wash step, an at least second material is applied or introduced, which fills up the spaces formed by the droplets of the first material.

This process, also described here as infiltration method or infiltration process, involves printing on the substrate one or more layers composed of individual droplets of the first material. Said droplets, which form corresponding regions, are preferably formed in predetermined regions. Usually, said droplets, or the structures formed by said droplets, are spaced apart, with the result that corresponding spaces are formed. If necessary, the printing materials are cured, for example by gelling or polymerization, such that the cells possibly present therein remain alive (vital).

After construction of the layers with the first material (the one material), a rinsing or a washing with a rinse liquid is optionally carried out. Thereafter, the second material (the other material) is applied; this can likewise be done by a printing process, but it is also alternatively possible to use pipetting, spraying or other application techniques.

Said second material is infiltrated into the spaces, where they form at least one second subregion. Said second material can likewise comprise cells and usually comprises a material other than the first printing material. After the application, the three steps of droplet printing, washing and infiltration can be repeated as often as necessary in order to produce multilayer constructs. Especially in the case of a rinsing operation (washing) between the application of the first material and the second material, it is possible to fill up the spaces between the first material particularly well owing to the capillary forces.

When suitable cell types (e.g., endothelial cells and mesenchymal stem cells) are used in the second material, which is infiltrated between the droplets of the first material, the described process makes it possible to form a directed capillary-like network. The growth of the capillary-like structure can be set a desired direction through the positioning of the droplets consisting of the first material.

This process for forming this structure can be followed by a further step, specifically a step in which the first material is leached from the three-dimensional structure formed. This means that, in the case of a hydrogel containing live cells, said hydrogel can, for example, be liquefied by appropriate heating and thus removed from said structure. It became apparent that, when removing this printing material, the cells possibly present therein adhere to the edges of the further subregion. By means of this process, it is particularly easily possible to form, for example, further supply structures (e.g., vascular structures) which run orthogonally to the printed structure. The removal of the material can create cavities, on the inner side of the outer boundary of which these cells are deposited. This method is suitable especially for the formation of vascular structures and epithelialized channels (e.g., tubules), in which cells forming vascular structures, such as endothelial cells, epithelial cells, mesenchymal stem cells, fibroblasts and/or smooth muscle cells and including combinations thereof, are introduced with the first material.

In a further embodiment, the penetration method, the procedure is in principle done in reverse order. This means that one material is applied to a substrate which has optionally been treated beforehand with a rinse solution. This application can be done by printing, pipetting, spraying or other application techniques. Said material can likewise comprise cells. This can be followed by, if necessary, a curing of said material so that the further material can then be printed by droplet printing onto this material film as individual droplets.

As a result of the printing, the droplet of the printing material can break through the surface of the film formed, for example such that the droplets are embedded in the layer formed by the material. Thus, the droplet dips into the other material and displaces it; what takes place is a penetration of the film by the droplets.

Through the introduction of said droplets into the layer formed as a film, it is likewise possible to form corresponding subregions. The droplets can be introduced at predetermined positions, with the result that the desired structure is formed. Here too, what can again optionally take place is a removal of one of these materials, for example by melting of the printing material. What can be formed as a result are especially structures having cavities, in which cells introduced by the printing material are accordingly deposited on the edges of said cavities.

In one embodiment, the droplets are printed in a predetermined pattern during the droplet printing. The droplets can be applied layer by layer in order to form three-dimensional structures of the subregions.

These substructures, either as a first subregion or as a second subregion, are accordingly embedded and are suitable especially for ensuring supply structures or microstructures for supplying or discharging nutrients, necessary metabolites including fluid, etc. Using the process according to the invention, it is accordingly possible to form microstructures and microorganizations of the tissues. At the same time, the process according to the invention allows, owing to the general applicability, the formation of a very wide variety of different tissue structures, which differ according to use of live cells, to the printing material in which they are situated and to the manner of introduction, for example by means of the infiltration method or the penetration method.

In one embodiment, the printing material is a material based on gelatin, especially a gelatin-agarose mix, polyethylene glycol (PEG) or a PEG derivative, or a poloxamer, such as pluronic, wherein said material can comprise live cells and/or wherein said material is liquefiable and removable at a later time, leaving the cells behind.

In one embodiment, the material which is applied by droplet printing is a stable or redissolvable hydrogel. Examples of a stable hydrogel of said printing material include agarose, alginate and other polysaccharides and also mixtures of these materials with protein-based gels (e.g., collagen, fibrinogen, etc.); redissolvable materials are, for example, gelatin or pluronic, a block copolymer composed of ethylene oxide and propylene oxide. Microgels, intramolecularly crosslinked macromolecules, can likewise be used as redissolvable materials.

By means of the dissolvable hydrogels, it is possible to form corresponding cavities in the three-dimensional biological structure. In one embodiment, this printing material contains cells which then, upon leaching, adhere to the edge of the cavities, where they can form desired structures, for example vascular structures or epithelialized tubules or other microstructures of the desired tissue.

These dissolvable droplets can form corresponding pores or cavities, with the result that a corresponding structure, for example in the form of a mesh or the like, is then formed from the at least one other material with defined pore size and interval.

Corresponding pores can be used in order to allow transport of the nutrients or other fluids, especially transport thereof to cells situated within or on the second material. Exemplary formations of such structures are described in the example.

The second or other material is preferably a hydrogel, especially one based on fibrinogen or collagen, which can optionally comprise cells. After application by generally known application processes, especially by printing, said material forms fibrin structures and/or collagen structures which are especially suitable for forming three-dimensional tissue structures which, for example, can be used even in the area of implants.

Suitable biocompatible materials are known to a person skilled in the art and are commercially available.

Owing to the individualized application of these at least two printing materials for the formation of the at least two subregions, it is possible to form individualized three-dimensional constructs which can be produced easily in large numbers and reproducibly.

In a further embodiment, the printing materials furthermore comprise chemical, biological or physical crosslinkers, which allow a corresponding curing of the material and optionally diffuse into the other material in order to initiate the gelling thereof.

Suitable materials are, for example, thrombin, calcium chloride, glutaraldehyde, genipin or transglutaminase.

Other suitable materials present in the printing materials can be chemical or biological or physical polymerization initiators or catalysts, which optionally diffuse into the other material and, contact therewith, bring about the gelling or solidification thereof. Suitable polymerization initiators or catalysts are known to a person skilled in the art, such as photoinitiators, photocatalysts, acids or alkaline solutions for adjusting the pH required for gelling, etc.

Suitable structures are known to a person skilled in the art.

In a further embodiment, the process is one for forming a three-dimensional biological structure, wherein said three-dimensional biological structure is formed similarly to a cardiac structure, a liver structure, a kidney structure, an alveolar structure, a skin structure, a cartilage structure, a bone structure with or without bone marrow, a neural structure or mixed forms thereof. Said structures are especially suitable as tissue substitutes or as tissue analogs in clinical research. Furthermore, these corresponding structures are also suitable as implants as substitutes for damaged tissue. Further tissue applications are especially also bones including bone marrow and also cartilage structures. Likewise, corresponding skin analogs with formed epidermis, dermis and subcutis can be depicted. By means of the process according to the invention, it is possible to produce a multiplicity of corresponding tissue and organ analogs easily on a relatively large scale.

The multicellular spheroids can either be directly coprinted in the droplets or be embedded in the resultant pores after the leaching of the droplets. What can be provided as a result are the constructs, specifically tissue analogs and tissue substitutes, that have a capillary network and copy complex structures.

In one embodiment, the structure is a pancreas analog or pancreas substitute. In this case, what can be introduced with the aid of droplets or into the pores are MCS or individual cells from different cells occurring in the pancreas, such as α-cells, β-cells, δ-cells, PP cells, ε-cells or MCS with entire islets of Langerhans or parts thereof.

In a further embodiment, the process according to the invention is one in which application of the at least further material is followed by a culturing of this structure in a suitable incubator.

A person skilled in the art is aware of the appropriate culturing conditions for culturing these printed three-dimensional biological structures. They are appropriately chosen such that the tissue structures are formed.

In one embodiment, the three-dimensional biological structure is an organ. Organs include a liver, a kidney, skin.

In a further embodiment, the live cells in the material are especially those which are or comprise vessel-forming cells, such as endothelial cells, epithelial cells, Schwann cells, nerve cells, mesenchymal stem cells, fibroblasts and/or smooth muscle cells.

In a further embodiment, the live cells in the printing material which forms the possibly remaining structure, such as the mesh or droplets embedded in the mesh, are cells such as hepatocytes, keratinocytes, nerve cells, (tubulo)epithelial cells and cardiomyocytes.

In a further embodiment, the cells are MCS as explained above. It is also possible to use mixtures of MCS and individual cells.

With the process according to the invention, specifically printing by means of droplets, it became apparent that the structures obtained have a particularly good dimensional stability and that printing is greatly improved.

With the process according to the invention, in which structures consisting of at least two subregions with at least two printing materials are printed, the use of these different printing materials and each of the hydrogels present therein allows a reciprocal stabilization of these regions, for example when using a less stable fibrin. Through the use of the different materials, it is possible to form corresponding organized capillary-like networks; for example, endothelial cells are suitable for forming these microorganized capillary-like networks in the subregions of the one printing material.

Various cell types and matrices can also be arranged in an appropriate manner according to tissue morphology with a high degree of cell functionality.

Suitable materials for the printing materials include agarose, collagen, fibrin, alginate, chitosan, hyaluronic acid, elastin/fibronectin-based hydrogels, hyaloronic acid or synthetic hydrogels including polyethylene glycol, poly(N-isopropylacrylamide) and copolymers, polylactides, polyurethanes or polyvinyl alcohols or mixtures thereof, poloxamers, such as, for example, pluronic, but also synthetically modified hydrogels including methacrylated gelatin (GelMA) or silicones.

Furthermore, the viscosity of the printing materials should be appropriately adjusted. In principle, no particular demands are put on the materials used, but the materials are preferably those which are dispensable. For example, gels can be printed up to approx. 5000 mPas by means of drop-on-demand printing; with other printing methods, viscosities of up to 960 000 mPas can also be printed.

Depending on the printing process, the viscosities of the at least two printing materials for the at least two printing subregions can be selected such that they are appropriately far apart, with the result that there is no mixing or only slight mixing.

In a further aspect, the printing materials are selected such that they exhibit a slight mixing owing to different hydrophobic properties. For example, after gelling or polymerization, the first material can form a surface on which subsequent droplets can be applied with a large contact angle, for example >20°. A person skilled in the art is aware of suitable hydrophobization agents and hydrophilic or hydrophobic materials that are used in the printing materials. Hydrophobicity, especially that of the first printing material, is important insofar as the droplets which are printed onto the hydrophobic material spread apart less greatly. As a result, a hydrophobic base layer or subsequently printed hydrophobic layers makes it possible to place distinctly more defined and spatially more highly resolved individual droplets and droplet structures. Furthermore, the intervals between individual droplets and droplet structures can also be set more precisely by printing onto a hydrophobic layer.

Lastly, in a further aspect, what is provided is a biological three-dimensional structure obtainable using the process according to the invention. In one embodiment, said biological three-dimensional structure is a tissue structure which can be used as a tissue substitute. Appropriate tissue substitutes are used in vitro as a tissue model, for example as a model for tissue genesis or as a tissue model for testing forms of therapy or for stratifying a therapy or for testing or identifying active-ingredient candidates.

Furthermore, these structures can be used as tissue substitutes in vivo, i.e., as an implant.

These tissue structures for in vitro or in vivo use can be organs or living-tissue structures, such as a capillary network, a liver tissue structure, a cardiac tissue structure, a kidney tissue structure, an alveolar structure, a skin structure, a cartilage structure, a bone structure with or without bone marrow, a neural structure or mixed forms thereof.

In one aspect, these tissue structures or tissue substitutes can also appropriately comprise MCS or combinations of MCS with individual cells in order to form the corresponding structures.

The culturing conditions for the formation of these tissue structures are known to a person skilled in the art; appropriate culturing processes can be carried out in appropriate devices with suitable materials. These structures according to the invention are notable in that they show an appropriate microstructure, especially with regard to the arrangement of the various cell types and matrices.

The invention will be further elucidated with reference to the figures and in the examples.

### EXAMPLES

The invention will be more particularly elucidated on the basis of the examples and the attached figures without being restricted thereto.

### Example 1

Formation of Tissues Containing Live Cells and Organs

In an extensive study, various tissue substitutes were produced using the process according to the invention. To this end, the following compositions were used: agarose (2%), gelatin (10%), fibrinogen (50 mg/ml), thrombin (100 units/ml), PBS, cell culture medium.

Depending on the desired tissue structure, the following cells were introduced into the printing material.

Preparation

For all the studies, a base layer is prepared by pouring 0.5 ml of gelatin-agarose solution (GA), consisting of equal parts of mixed agarose (2%) and gelatin (10%) enriched with 2% thrombin, into a Petri dish or a 12-well plate.

Production of Networks having Capillary-Like Structures (Infiltration)

With the aid of a drop-on-demand bioprinter (e.g., from Black Drop Biodrucker GmbH), a pattern of spaced individual droplets consisting of a GA mixture as described above is printed onto the base layer. The printing process is carried out at an air pressure of 0.5 bar, a valve opening time of 450 μs, and a valve diameter of 300 μm. The printing operation is repeated once. Thereafter, the droplet structures are washed with 500 μl of PBS. Afterwards, for each sample, 30 μl of cell-loaded fibrinogen (3×10̂6 HUVECs per ml and 1×10̂6 hMSCs per ml) are pipetted, printed or sprayed onto the printed sample. Printing operation and infiltration can be repeated as often as desired in order to construct multilayer structures. The samples are then cultured at 37° C. and 5% CO2 over a period of at least two weeks.

Production of Networks having Capillary-Like Structures (Penetration)

The base layer is washed with 500 μl of PBS. Afterwards, for each sample, 30 μl of cell-loaded fibrinogen (3×10̂6 HUVECs per ml and 1×10̂6 hMSCs per ml) are pipetted, printed or sprayed onto the base layer. Thereafter, a pattern of spaced individual droplets of an agarose mixture, consisting of equal parts of mixed agarose (2%) and cell culture medium enriched with 2% thrombin, is printed onto the fibrin-coated base layer with the aid of a drop-on-demand bioprinter (e.g., from Black Drop Biodrucker GmbH). Coating and printing operation can be repeated as often as desired in order to construct multilayer structures. The samples are then cultured at 37° C. and 5% CO2 over a period of at least two weeks.

Production of Cardiac Muscle Precursor Tissue (Infiltration)

With the aid of a drop-on-demand bioprinter (e.g., from Black Drop Biodrucker GmbH), a pattern of spaced individual droplets consisting of a GA mixture as described above is printed onto the base layer. The printing process is carried out at an air pressure of 0.5 bar, a valve opening time of 450 s, and a valve diameter of 300 μm. The printing operation is repeated once. Thereafter, the droplet structures are washed with 500 μl of PBS. Afterwards, for each sample, 30 μl of cell-loaded fibrinogen (3×10̂6 cardiomyocytes) are pipetted, printed or sprayed onto the printed sample. Printing operation and infiltration can be repeated as often as desired in order to construct multilayer structures. The samples are then cultured at 37° C. and 5% CO2 over a period of at least 5 days.

Production of Cardiac Muscle Precursor Tissue (Penetration)

The base layer is washed with 500 μl of PBS. Afterwards, for each sample, 30 μl of cell-loaded fibrinogen (3×10̂6 cardiomyocytes) are pipetted, printed or sprayed onto the base layer. Thereafter, a pattern of spaced individual droplets of an agarose mixture, consisting of equal parts of mixed agarose (2%) and cell culture medium enriched with 2% thrombin, is printed onto the fibrin-coated base layer with the aid of a drop-on-demand bioprinter (e.g., from Black Drop Biodrucker

GmbH). Coating and printing operation can be repeated as often as desired in order to construct multilayer structures. The samples are then cultured at 37° C. and 5% CO2 over a period of at least 5 days.

Production of Kidney Analog (Infiltration)

With the aid of a drop-on-demand bioprinter (e.g., from Black Drop Biodrucker GmbH), a pattern of spaced individual droplets consisting of a GA mixture as described above, provided with 3×10̂6 HK-2 tubuloepithelial cells per milliliter, is printed onto the base layer. The printing process is carried out at an air pressure of 0.5 bar, a valve opening time of 450 s, and a valve diameter of 300 μm. The printing operation is repeated once. Thereafter, the droplet structures are washed with 500 μl of PBS. Afterwards, for each sample, 30 μl of cell-loaded fibrinogen (3×10̂6 HUVECs per ml and 1×10̂6 hMSCs per ml) are pipetted, printed or sprayed onto the printed sample. Printing operation and infiltration can be repeated as often as desired in order to construct multilayer structures. The samples are then cultured at 37° C. and 5% CO2 over a period of at least 1 week.

Production of Liver Lobe Analog (Penetration)

The base layer is washed with 500 μl of PBS. Afterwards, for each sample, 30 μl of cell-loaded fibrinogen (3×10̂6 HUVECs per ml and 1×10̂6 hMSCs per ml) are pipetted, printed or sprayed onto the base layer. Thereafter, a pattern of spaced individual droplets of an agarose mixture, consisting of equal parts of mixed agarose (2%) and cell culture medium enriched with 2% thrombin, is printed onto the fibrin-coated base layer with the aid of a drop-on-demand bioprinter (e.g., from Black Drop Biodrucker GmbH). The agarose hydrogel used for printing is hepatocyte-loaded (3×10̂6 HUH7 hepatocytes). Coating and printing operation can be repeated as often as desired in order to construct multilayer structures. As control, a sample which contained no fibrin and no hMSCs and HUVECs present therein was produced in the same manner. The samples are then cultured at 37° C. and 5% CO2 over a period of at least 12 days.

Evaluation

The survival rate of the cells situated in the printed structures was evaluated by carrying out vital-fluorescence twin stainings (FDA/PI). The structures were analyzed with the aid of a light microscope with scan function at 5-times and 10-times magnification. The capillary-like network formation was depicted by fixing the samples with methanol and labeling them with DAPI and an antibody stain specific for endothelial cells (CD31). The tubuloepithelial cells were additionally labeled with an antibody stain specific for their part (LTL). The samples were analyzed with the aid of a fluorescence microscope with scan function and by means of two-photon microscopy at 5-times, 10-times and 20-times magnification. The synthesis of urea in the liver lobe models was determined by taking, in each case, 500 μl of supernatant from the samples on day 4, 8 and 12 of culturing and subjecting said supernatant to a photometric measurement. A distinction was made between the samples containing hepatocytes and fibrin and also the hMSCs and HUVECs present therein (HEP+), the samples containing only hepatocytes (HEP) and the cell culture medium (Control).

According to the invention, what is provided is a universal process for the biofabrication of tissue substitutes and tissue analogs. Using said universal process, it is possible to provide a multiplicity of tissues having complex structures which, as described in the examples, reflect the original tissues very well. Said tissues are accordingly suitable as substitutes, for example as implants, but also as analogs for in vitro methods, for example in the pharmaceutical sector. Owing to its simplicity, the process according to the invention allows a rapid production in a high number of tissues and permits the production of substantially identical tissue structures.

1. A process for bioprinting a three-dimensional biological structure containing live cells, comprising:
first applying or introducing to a substrate a first material, said first material being optionally subjected to a first treatment after the first applying or introducing step; and then
second applying or introducing to the substrate a second material where the first material has been applied or introduced, wherein the second material is different from the first material,
wherein the first applying or introducing and the second applying or introducing step produce the three-dimensional biological structure which has
at least two different subregions, wherein at least one subregion of the three-dimensional structure contains live cells, and wherein at least one of the first material or second material contains live cells, and wherein at least one of the first material or the second material is applied or introduced by droplet printing.

2. The process as claimed in claim 1, wherein the first material is printed onto the substrate as droplets in the first applying or introducing step, wherein the droplets are deposited at predetermined positions in relation to one another with spaces therebetween, and wherein the second material is applied or introduced so as to fill up the spaces formed by the droplets of the first material.

3. The process as claimed in claim 1, wherein, in the second applying or introducing step the second material is printed into the first material as droplets such that the second material, when printed, dips into the first material or displaces it at least in part.

4. The process as claimed in claim 3, wherein the printed droplets of the second material breaks through a layer of the first material such that the droplets of the second material are embedded in the layer formed by the first material.

5. The process as claimed in claim 1 4, wherein the droplets are printed in a predetermined pattern during the droplet printing.

6. The process as claimed in claim 1 wherein the first material is a material based on gelatin, polyethylene glycol (PEG) or a PEG derivative, or a poloxamer, wherein the first material comprises the live cells and wherein the first material is optionally liquefiable and removable at a later time, leaving the cells behind.

7. The process as claimed in claim 1 wherein the second material is a hydrogel, which optionally contains the living cells.

8. The process as claimed in claim 1 wherein the first material is mixed with a chemical or biological or physical crosslinker, which optionally diffuses into the second material and, upon contact with the second material, brings about the gelling of the second material.

9. The process as claimed in claim 1 wherein the three-dimensional structure is a tissue structure having a first region which corresponds to a supply structure and a second region which forms a functional tissue.

10. The process as claimed in claim 1 wherein the three-dimensional structure resembles a cardiac structure, a liver structure, a kidney structure, an alveolar structure, a skin structure, a cartilage structure, a bone structure with or without bone marrow, a neural structure or mixed forms thereof.

11. The process as claimed in claim 1, further comprising culturing the three-dimensional structure after the second applying or introducing step in an incubator.

12. The process as claimed in claim 1 wherein the three-dimensional structure is an organ.

13. The process as claimed in claim 1 wherein the live cells are in the first material and are or comprise vessel-forming cells, mesenchymal stem cells, fibroblasts and/or smooth muscle cells.

14. A biological three-dimensional structure produced by the process of claim 1.

15. The biological three-dimensional structure as claimed in claim 14, wherein the three-dimensional structure is a liver tissue structure, a cardiac tissue structure, a kidney tissue structure, an alveolar structure, a skin structure, a bone structure with or without bone marrow, a cartilage structure, a neural structure or mixed forms thereof.

16. A method of using the three-dimensional structure as claimed in claim 14 as a model for tissue genesis.

17. A method of using the three-dimensional structure as claimed in claim 14 as a tissue model for testing forms of therapy or for stratifying a therapy or for testing or identifying active-ingredient candidates.
